(19) United States
(12) Patent Application Publication
(10) Pub. No.: US 2002/0173522 A1
(43) Pub. Date: Nov. 21, 2002

(54) Pharmaceutical compositions comprising norastemizole

(76) Inventors: Martin P. Redmon, Oxford, MA (US); Hal T. Butler, Marlborough, MA (US); Stephen A. Wald, Sudbury, MA (US)

Correspondence Address: PENNIE & EDMONDS LLP, 1667 K STREET NW, SUITE 1000, WASHINGTON, DC, 20006

(21) Appl. No.: 10/075,616

(22) Filed: Feb. 15, 2002

Related U.S. Application Data
(63) Continuation-in-part of application No. 09/719,843, filed on Nov. 21, 2000, now abandoned.
Application No. 09/719,843 is a 371 of international application No. PCT/US98/05701, filed on Mar. 25, 1998.
Application No. PCT/US98/05701 is a continuation-in-part of application No. 08/851,786, filed on May 6, 1997, now abandoned.
Application No. 08/851,786 is a continuation-in-part of application No. 08/824,477, filed on Mar. 26, 1997, now abandoned.
Application No. 08/851,786 is a continuation-in-part of application No. 09/721,711, filed on Nov. 27, 2000, now abandoned.
Application No. 09/721,711 is a continuation-in-part of application No. 08/851,786, filed on May 6, 1997, now abandoned.
Application No. 08/851,786 is a continuation-in-part of application No. 08/824,477, filed on Mar. 26, 1997, now abandoned.

(51) Int. Cl. A61K 31/454

(52) U.S. Cl. 514/322

(57) ABSTRACT

The present invention relates to chemically stable pharmaceutical formulations of the potent antihistamine, norastemizole. The compositions can include norastemizole, or a pharmaceutically acceptable salt thereof; a diluent; a binder; a disintegrant; and a lubricant or the compositions can include particles of norastemizole, or a pharmaceutically acceptable salt thereof, coated with an inert coating and a pharmaceutically acceptable excipient. The present invention also relates to methods of treating allergic disorders.

### CROSS-REFERENCE TO RELATED APPLICATIONS

This application is a continuation-in-part of pending application Ser. No. 09/719,843, filed Nov. 21, 2000, which is a 371 of PCT/US98/05701, filed Mar. 25, 1998, which is a continuation-in-part of pending application Ser. No. 08/851,786, filed May 6, 1997, which is a continuation-in-part of pending application Ser. No. 08/824,477, filed Mar. 26, 1997; this application is also a continuation-in-part of pending application Ser. No. 09/721,711, filed Nov. 27, 2000, which is a continuation-in-part of pending application Ser. No. 08/851,786, filed May 6, 1997, which is a continuation-in-part of pending application Ser. No. 08/824,477, filed Mar. 26, 1997, all of which are expressly incorporated herein by reference thereto in their entirety.

### FIELD OF THE INVENTION

The present invention relates to chemically and thermally stable pharmaceutical compositions containing norastemizole.

### BACKGROUND OF THE INVENTION

Many factors affect the stability of a pharmaceutical product, including the stability of the therapeutic drug ingredient(s), the potential interaction between the therapeutic drug ingredient(s) and the inactive ingredient(s), the manufacturing process, the packaging, the environmental conditions encountered during shipment, storage and handling, the length of time between manufacture and usage and the type of the dosage form. In addition to physical stability, the chemical stability of the pharmaceutical product should be considered. Knowledge of the physical and chemical stability of a pharmaceutical formulation is very important for at least three primary reasons.

First, a pharmaceutical product, preferably, should appear fresh, elegant and professional. Any changes in physical appearance and color including fading, color variation, appearance of haziness and the like can cause the patient to lose confidence in the product. Second, since some products are dispensed in multiple-dose containers, uniform dosage of the therapeutic agent(s) over time must be assured. For example, a non-uniform dosage pattern may be indicated by a cloudy solution, a broken emulsion, a discolored tablet, a discolored capsule or the like. Third, the therapeutic drug ingredient(s) must be available to the patient throughout the expected shelf life of the dosage form. A breakdown in the physical or chemical integrity of the dosage form can lead to a lack of bioavailability or detrimentally altered bioavailability of the therapeutic drug ingredient(s).

A variety of pharmaceutical dosage forms are available for successfully administering many marketed drugs. Common pharmaceutical dosage forms listed in the U.S. Pharmacopeia/National Formulary (USP/NF) include, but are not limited to, aerosols, capsules, cachets, collyria, creams, emulsions, extracts, fluid extracts, gels, inhalations, injections, lotions, magmas, milks, ointments, pastes, pellets or implants, powders, solutions, ophthalmic solutions, oral solutions, otic solutions, pastilles, topical solutions, spirits, suppositories, suspensions, sublingual lozenges, syrups, tablets, tinctures, troches, aromatic waters and the like. For oral administration, syrups, solutions, suspensions, troches, tablets and capsules are preferred. However, for greater ease of administration, for increased carrying convenience and for improved patient compliance with a prescribed dosage regimen, troches, tablets, and hard and soft gelatin capsules are most preferred. In some cases, tablets are preferred over capsules because tablets are sometimes easier to swallow.

Troches, tablets and capsules, typically, contain the drug ingredient, a diluent and other excipients, such as lubricants and the like, which are well known in the art. Well known excipients include, for example, coating agents, colorants, desiccants, emulsifying agents, solubilizing agents, flavors, anti-caking agents, plasticizers, suspending agents, viscosity increasing agents, binders, diluents, wetting agents and the like.

Lactose is a commonly used diluent or excipient. Spray-dried lactose is a commonly available form of lactose which is widely used as a direct compression excipient. Since the advent of spray-dried lactose, its use as an excipient has expanded. The rapid acceptance of spray-dried lactose is, in part, due to its ease of incorporation in direct compression tablets. In this application, spray-dried lactose is in its ready-to-use form and does not require further granulation or introduction of complicated processing steps. Spray-dried lactose can also be readily and conveniently incorporated into a troche or a capsule dosage form. Spray-dried lactose may be directly added to a drug to yield a desired dilution ratio therewith. Thereafter, for example, the combination of the lactose and the drug may be dry compressed into a tablet or formulated into a troche or a capsule with other excipients, as necessary.

Lactose, whether spray-dried or not, is typically present in equilibrium between its alpha and beta forms, wherein interconversion between these forms is ongoing. Alpha-lactose is a disaccharide of beta-D-galactose and alpha-D-glucose. Beta-lactose is a disaccharide of beta-D-galactose and beta-D-glucose. Beta-lactose occurs only in its anhydrous form, whereas alpha-lactose may be obtained either in anhydrous form or as a monohydrate.

During interconversion between the alpha and beta forms of lactose, an aldehyde intermediate is formed which is known to be incompatible with most primary amines. Primary amines add to the carbonyl carbon of aldehydes (and ketones) to form imines:

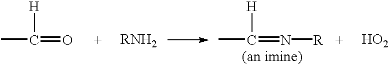

The incompatibility of most primary amines with lactose is well-recognized. See, Castello et al., J. Pharm. Sci., 51 (2):106-108 (Feb. 1962). See also, Blaug et al., J. Pharm. Sci., 61(11):1770-1775 (Nov. 1972); Hartauer et al., Drug Dev. and Indust. Pharm., 17(4):617-630 (1991).

Castello et al. tested the compatibility of amphetamine sulfate (a primary amine salt) with lactose. They found that a mixture of lactose and amphetamine sulfate became discolored, especially in the presence of alkaline lubricants such as magnesium stearate. Blaug et al. tested dextroamphetamine sulfate (a primary amine salt) with spray-dried lactose. They found that the lactose formed a Schiff base (i.e., an imine) in the presence of dextroamphetamine sulfate. Hartauer et al. tested aminophylline with lactose, and found that some incompatibility, evidenced by discoloration, between aminophylline and lactose occurred, especially when heat, of about 60° C., was applied. Aminophylline contains a ratio of two molecules of theophylline (a secondary amine) for one molecule of ethylene diamine (a primary amine). However, Hartauer et al. tested these components and found that while theophylline alone (a secondary amine) did not react with lactose in the presence or absence of heating to 60° C., ethylene diamine did react with the lactose, especially when heated to 60° C. Thus, the incompatibility of aminophylline with lactose appeared to result from incompatibility of the primary amine component of aminophylline, ethylenediamine, with lactose.

The drug astemizole, a secondary amine, appears to be compatible with lactose, as it is commercially available as HISMANAL® in a tablet dosage form containing lactose. According to the Physician's Desk Reference, 50th Edition, Medical Economics Co., Montvale, N.J., p. 1293 (1996), each tablet of Hismanal® contains 10 mg astemizole, lactose, cornstarch, microcrystalline cellulose, pre-gelatinized starch, povidone K90, magnesium stearate, colloidal silicon dioxide and sodium lauryl sulfate.

Likewise, it is expected that norastemizole, another secondary amine and the primary metabolite of astemizole, should be compatible with lactose, especially in the absence of applied heat. Norastemizole has been reported to be both more potent and less toxic than astemizole. Thus, norastemizole is an attractive alternative to astemizole for the treatment of allergic disorders. It should be recognized that both astemizole and norastemizole are antihistamines containing secondary amine moieties; however, norastemizole has two secondary amine moieties, whereas astemizole has one.

### SUMMARY OF THE INVENTION

The present invention relates to stable solid pharmaceutical dosage forms of norastemizole. In one embodiment the solid pharmaceutical composition comprises norastemizole, or a pharmaceutically acceptable salt thereof; a diluent; a binder; a disintegrant; and a lubricant; wherein the diluent, binder, disintegrant, and lubricant are not the same. The pharmaceutical composition can be free of lactose. The disintegrant can be present in an amount of from about 0.5 to 15 weight percent of the composition, and the lubricant can be present in an amount of up to about 1 weight percent of the composition. The composition can be in a tablet or capsule dosage form. The norastemizole, or a pharmaceutically acceptable salt thereof, can be present in an amount of from about 1 mg to 200 mg. In one embodiment the norastemizole, or a pharmaceutically acceptable salt thereof, is present in an amount of about 2 mg to 100 mg. The pharmaceutical composition can further comprise a decongestant such as, but not limited to, pseudoephedrine, or a pharmaceutically acceptable salt thereof. The decongestant, or a pharmaceutically acceptable salt thereof, can be adapted for sustained release.

In a second embodiment the solid pharmaceutical composition comprises norastemizole, or a pharmaceutically acceptable salt thereof; microcrystalline cellulose; pregelatanized starch; croscarmellose sodium; and magnesium stearate. The norastemizole, or a pharmaceutically acceptable salt thereof, can be present in an amount of from about 1 to 50 percent; the microcrystalline cellulose can be present in an amount of from about 20 to 90 percent; the pregelatanized starch can be present in an amount of from about 5 to 75 percent; the croscarmellose sodium can be present in an amount of from about 1 to 5 percent; and the magnesium stearate can be present in an amount of from about 0.05 to 0.8 percent by weight of the pharmaceutical composition. The pharmaceutical composition can be a tablet or capsule dosage form. The norastemizole, or a pharmaceutically acceptable salt thereof, can be present in an amount of from about 1 mg to 200 mg. In one embodiment, the norastemizole, or a pharmaceutically acceptable salt thereof, is present in an amount of about 2 mg to 100 mg. The pharmaceutical composition can further comprise a decongestant such as, but not limited to, pseudoephedrine, or a pharmaceutically acceptable salt thereof. The decongestant, or a pharmaceutically acceptable salt thereof, can be adapted for sustained release.

In a third embodiment, the solid pharmaceutical composition comprises (i) a therapeutically effective amount of coated particles of norastemizole, or a pharmaceutically acceptable salt thereof, wherein said particles are coated with an inert coating and (ii) a pharmaceutically acceptable excipient. The coated particles of norastemizole, or a pharmaceutically acceptable salt thereof, can comprise granulated norastemizole particles, or a pharmaceutically acceptable salt thereof, and a pharmaceutically acceptable excipient. The inert coating can comprise an inert film-forming agent in a solvent. Examples of inert film-forming agents include, but are not limited to, methylcellulose, hydroxymethyl cellulose, carboxymethyl cellulose, hydroxypropylmethylcellulose, hydroxypropyl cellulose, hydroxyethylcellulose, methylhydroxyethylcelluose, sodium carboxymethylcellulose, and mixtures thereof. Another example of an inert film-forming agent is cross-linked ethylcellulose. The solid pharmaceutical composition can be adapted as a quick dissolving dosage form.

In a fourth embodiment, the solid pharmaceutical composition comprises norastemizole, or a pharmaceutically acceptable salt thereof; a diluent; a binder; a disintegrant; and a lubricant; wherein the disintegrant is a super disintegrant. Examples of super disintegrants include, but are not limited to, croscarmellose sodium and sodium starch glycolate.

The present invention is also directed to a method of treating allergic disorders in a mammal comprising administering to said mammal a therapeutically effective amount of a composition of the invention. The mammal can be a human. Examples of allergic disorder include, but are not limited to, allergic rhinitis, solar uticaria, or symptomatic dermographism.

### BRIEF DESCRIPTION OF THE DRAWINGS

FIG. 1 depicts the chemical structure of norastemizole.

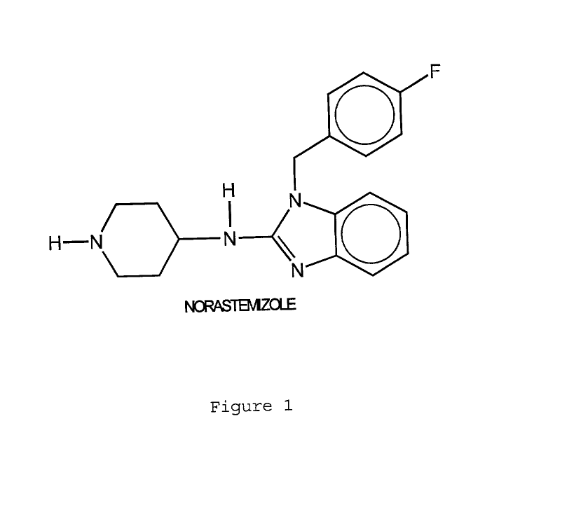

FIG. 2 presents in bar-graph format the change in initial potency of a dosage form of norastemizole and various pharmaceutical excipients when the dosage form is exposed to a temperature of 60° C. at 75% relative humidity using non-hermetically sealed containers (i.e., screw-top vials).

### DETAILED DESCRIPTION OF THE INVENTION

Applicants have discovered that, even in the absence of applied heat, surprisingly, the discoloration reaction found with primary amines and lactose is also found with norastemizole. Thus, there appears to be an heretofore unappreciated incompatibility between the secondary amine, norastemizole, and lactose. It is, therefore, desirable to formulate dosage forms of norastemizole that are lactose-free. Further, Applicants have also discovered that the instability of lactose and norastemizole may be initiated and/or accelerated upon the exposure of a norastemizole/lactose formulation to water, including atmospheric moisture, e.g., humidity. The instability is also initiated and/or accelerated upon exposure to heat at temperatures of greater than about 60° C. Moreover, Applicants have also discovered that the instability of lactose and norastemizole may be initiated and/or accelerated by the high surface area of the small particles of norastemizole conventionally used in pharmaceutical compositions upon the exposure of a norastemizole/lactose formulation. Additionally, Applicants have also discovered that the instability of lactose and norastemizole may be inhibited or avoided by coating norastemizole particles prior to formulation of the norastemizole with reactive excipients, such as lactose.

In PCT application PCT/US93/08349, published as WO 94/07495, a formulation of norastemizole is proposed in Example 4, which happens to lack lactose. Formulas A, B and C, of Example 4, each contain 1.0 weight percent of magnesium stearate BP, 94.0, 89.0 and 79.0 weight percent of Starch 1500 (a pre-gelatinized starch commercially available from Colorcon, Ltd.), respectively, and the remainder of the composition is a metabolite of astemizole (e.g., norastemizole). However, this publication neither discloses nor suggests that norastemizole and lactose are incompatible, as evidenced by the lactose-containing tablet formulation of norastemizole in Example 5 therein.

In view of the heretofore unappreciated problems associated with pharmaceutical formulations including the secondary amine, norastemizole, and lactose, it is desired to prepare stable solid pharmaceutical formulations of norastemizole that avoid the incompatibility between norastemizole and lactose. The present invention advantageously recognizes and provides lactose-free dosage formulations of norastemizole.

Based upon the pharmacological benefits of norastemizole over astemizole, there is a need for stable, high performance dosage forms of norastemizole. To date, there is no commercially available stable norastemizole formulation. However, the inventors have found that by eliminating lactose and using the alternative ingredients described herein, lactose-free dosage forms of norastemizole are surprisingly chemically, physically and thermally stable. This stability may be achieved by the present invention without loss of either manufacturing ease or dosage performance.

One feature of the present invention is thus directed to chemically and thermally stable pharmaceutical formulations that include norastemizole, or a pharmaceutically acceptable salt thereof, and a pharmaceutically acceptable carrier or excipient that does not include or utilize any form of lactose. Lactose has been widely accepted and used by the pharmaceutical industry, inter alia, because of its ease of manufacture. However, Applicants have advantageously found that formulations containing norastemizole and lactose are unstable over time and degrade more rapidly upon exposure to heat and moisture.

Secondary amines were previously considered to be compatible with lactose, especially at ambient temperatures or where exposure to heat (e.g., below about 60° C.) is either minimal or altogether avoided. As noted, for example, the drug astemizole is available in a tablet dosage form containing lactose and other excipients under the tradename Hismanal®.

It has now been discovered that physical and/or chemical incompatibility exists between the secondary amine, norastemizole, and lactose. Without being limited by theory, it is believed that the incompatibility of norastemizole with lactose results from the formation of enamines due to reaction between the aldehyde intermediate of lactose and a secondary amine:

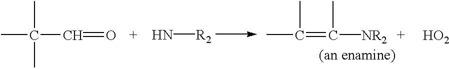

It has also been discovered that the incompatibility exists even at ambient temperatures (e.g., temperatures below about 60° C.) and at ambient relative humidity. Further, Applicants have also discovered highly stable pharmaceutical compositions containing norastemizole without the use of the widely accepted excipient lactose.

According to one feature of the present invention, norastemizole is provided in lactose-free pharmaceutical compositions. These compositions possess potent antihistaminic activity and are useful in treating a variety of conditions. Some of these conditions include, for example, allergic rhinitis, asthma, solar uticaria, symptomatic dermographism, and other allergic disorders, vertigo, motion sickness, vestibular disturbances (e.g., Meniere's disease), diabetic retinopathy, other small vessel disorders associated with diabetes melitis.

More importantly, these lactose-free compositions provide a stable and convenient dosage form for delivering norastemizole to humans. The lactose-free compositions of the invention are stable, inter alia, in that they have significant shelf-life. Further, the compositions of the invention remain stable even when exposed to mild temperature and humidity changes. Moreover, even though the compositions of the invention are lactose-free, the compositions are still easily manufactured, and the compositions have desirable dosage performance properties. The compositions of the invention include solid unit dose formulations comprising norastemizole, or a pharmaceutically acceptable salt thereof, and at least one non-lactose pharmaceutically acceptable excipient. The compositions may also optionally include other therapeutic ingredients, binders/fillers, disintegrants, lubricants, anti-caking agents, preservatives, film coating agents, sweetening agents, colorants, flavors, desiccants, plasticizers, dyes, dispersing agents and/or surface active agents. However, any such optional ingredient must be compatible with norastemizole, a secondary amine, to insure the stability of the formulation.

It is preferred that the lactose-free dosage form of norastemizole made in accordance with the present invention comprise norastemizole and at least one non-lactose excipient. Examples of such excipients are well known in the art and are listed in the USP (XXI)/NF (XVI), incorporated herein in its entirety by reference thereto. It is further preferred that the lactose-free norastemizole dosage forms made in accordance with the present invention comprise norastemizole, a binder/filler and a lubricant in pharmaceutically compatible and pharmaceutically acceptable amounts. It is even further preferred that the lactose-free norastemizole dosage forms made in accordance with the present invention comprise norastemizole, microcrystalline cellulose, pre-gelatinized starch, and magnesium stearate.

It has also been discovered that other sugars, such as fructose and sucrose, cause similar, although not as severe, degradation to that caused by lactose when used in combination with norastemizole containing formulations. Thus, in another embodiment, the lactose-free pharmaceutical compositions comprise norastemizole, or a pharmaceutically acceptable salt thereof, and at least one non-lactose pharmaceutically acceptable excipient, and do not contain any mono- or di-saccharide excipients, including, but not limited to, glucose, sucrose, and fructose.

As mentioned above, norastemizole formulations containing lactose that are exposed to unbound water, e.g., moisture or humidity, degrade more rapidly. The addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and temperature accelerate the degradation.

Further, the effect of water on a formulation is of reat significance since conditions favorable for ygroscopicity, e.g., moisture and/or humidity, are commonly encountered during manufacture, handling, packaging, storage, shipment and use of the formulation. Thus, it is clear that the use of lactose in pharmaceutical compositions or formulations containing norastemizole should be avoided due to the substantial contact with moisture and/or humidity that the compositions have under normal manufacturing, packaging and storage conditions.

Moreover, although excipients other than lactose may be readily used to manufacture the disclosed lactose-free pharmaceutical compositions of norastemizole without impacting on the manufacturability and therapeutic performance of the compositions, spray-dried lactose continues to be an excipient of choice. In the spray-dried form, lactose is among the best of all direct compression fillers in fluidity and is very effective for low dose formulations (e.g., ≦50 mg per dose) where the compactibility of the active ingredient does not play a major role in the formulation. See, e.g., R. Shangraw, Selection of Manufacturing Process and Excipients with an Emphasis on Direct Compression, Course material from Granulation, Tableting, and Capsule Technology, Center for Professional Advancement, East Brunswick, N.J., 1996. Therefore, when possible, it is desirable to include lactose among the available possible excipients for the solid dosage forms or pharmaceutical composition of norastemizole.

Therefore, as an alternative, the present invention encompasses thermally and chemically stable pharmaceutical compositions, particularly, solid pharmaceutical formulations, which comprise norastemizole, or a pharmaceutically acceptable salt thereof, and optionally one or more pharmaceutically acceptable excipients, including but not limited to lactose, wherein the lactose containing formulations are anhydrous, i.e., substantially free of unbound water.

The invention further encompasses thermally and chemically stable non-hygroscopic pharmaceutical compositions which comprise norastemizole, or a pharmaceutically acceptable salt thereof, and one or more excipients or ingredients including, but not limited to, lactose. Without being limited by any theory, these stable anhydrous or non-hygroscopic pharmaceutical compositions are based, in part, on Applicants' discovery that the incompatibility between norastemizole and lactose, or other mono-or di-saccharides, is accelerated and/or possibly initiated by exposure of such formulations to unbound water. Thus, preparing pharmaceutical compositions that are substantially free of unbound water will prevent the accelerated degradation of norastemizole that occurs when a reactive excipient is used and unbound water is present.

Thus, if lactose is a desired excipient, another aspect of the invention relates to non-hygroscopic or anhydrous pharmaceutical compositions comprising norastemizole, lactose and optionally one or more additional excipients or ingredients wherein the resulting pharmaceutical compositions are substantially free of unbound water. It should be recognized that the non-hygroscopic or anhydrous formulations can be made by standard methods, provided that suitable excipients are selected such that the resulting pharmaceutical compositions are substantially free of unbound water, and processing is conducted using conditions of low humidity.

Anhydrous norastemizole pharmaceutical composition prepared in accordance with the present invention should be prepared and stored such that the anhydrous nature is maintained. Accordingly, these compositions will be packaged using materials well known in the art for preventing exposure of the pharmaceutical composition to water, allowing them to be included in suitable formulary kits. Such packaging will include, but not be limited to, hermetically sealed foils, plastic or the like, unit dose containers, blister packs, or trip packs.

Accordingly, a second alternative aspect of the invention encompasses a method of preparing a solid harmaceutical formulation comprising norastemizole and lactose which method comprises admixing under anhydrous or low moisture/humidity conditions, norastemizole, or a harmaceutically acceptable salt thereof, and lactose wherein said ingredients are substantially free of unbound water. The method may optionally further comprise packaging said anhydrous or non-hygroscopic solid norastemizole formulation under low moisture conditions. By using such conditions, the risk of contact with water is reduced and the degradation of norastemizole is prevented or substantially reduced during processing and storage. Further, the final packaged product has little or no unbound water present which substantially improves stability and prevents degradation. Such compositions can be provided in hermetically sealed packages such as vials, sealed packets, blister packs and other vacuum sealed and moisture free containers well known to the skilled artisan.

Traditionally, when pharmaceutical compositions or formulations are prepared, the active ingredient or therapeutic agent (e.g., norastemizole) is milled and/or screened to decrease the particle size and/or narrow the particle size distribution. Most often, this is done in order to optimize various physicochemical characteristics of the formulation, such as dissolution, content uniformity, bioavailability of the active ingredient, and the like. Dissolution is of particular concern with norastemizole, since the solubility is relatively low (approximately 10 mg/mL) at pH 3-4 and lower above pH 4. Without being limited by any particular theory, however, Applicants believe that the interaction between norastemizole and reactive excipients, such as lactose, may be affected by the surface area of the norastemizole particles in the pharmaceutical composition or formulation.

Accordingly, another embodiment of the present invention encompasses pharmaceutical compositions for the treatment of histamine-induced disorders which comprise large particles of norastemizole, or pharmaceutically acceptable salts thereof, and a pharmaceutically acceptable carrier. Pharmaceutically acceptable carriers suitable for use in these compositions include carriers that may comprise one or more excipients selected from the group consisting of inert excipients and reactive excipients, such as lactose or other mono- or di-saccharides. These “large particle” pharmaceutical compositions of norastemizole have suitable physicochemical characteristics (in terms of dissolution, content uniformity, bioavailability, and the like), but do not exhibit incompatibility with reactive recipients, such as lactose.

In a preferred embodiment, the norastemizole, or a pharmaceutically acceptable salt thereof, present in the composition has a particle size distribution in which about 40% by weight or more of norastemizole, or a pharmaceutically acceptable salt thereof, comprises particles having a size of 200 μm or larger, preferably greater than about 250 μm.

Another means for inhibiting or preventing the interaction between norastemizole and reactive excipients, such as lactose, in a pharmaceutical composition is to prevent norastemizole from coming into contact with any reactive excipients in the composition. One manner in which this may be achieved is to coat the norastemizole particles with an inert or non-reactive coating prior to formulation with reactive excipients. Preferably, the inert coating should not significantly influence the pharmacodynamic characteristics (e.g., time to onset of efficacy, and absorption in vivo) of the composition. Advantageously, the inert coating also masks the bitter taste that can accompany orally administered norastemizole.

Accordingly, another embodiment of the present invention relates to solid pharmaceutical compositions for the treatment of histamine-induced disorders comprising a therapeutically effective amount of coated norastemizole, or a pharmaceutically acceptable salt thereof, which comprises norastemizole, or a pharmaceutically acceptable salt thereof, coated with an inert coating agent, and a pharmaceutically acceptable carrier. In a preferred embodiment, the norastemizole, or a pharmaceutically acceptable salt thereof, is first granulated with an inert excipient (e.g., starch), and then the resulting granules are coated with an inert or non-reactive coating agent. Thereafter, the resulting coated norastemizole may be blended with other excipients, including reactive excipients.

Suitable inert coating agents, and methods for coating particles or granules, are well known in the art. Inert coating agents typically comprise an inert film-forming agent dispersed in a suitable solvent, and may further comprise other pharmaceutically acceptable adjuvants, such as colorants and plasticizers. Preferably, the particles or granules of norastemizole are coated using aqueous or non-aqueous film coating techniques or microencapsulation. Suitable inert film-forming agents include, but are not limited to, celluloses, such as methylcellulose, hydroxymethyl cellulose, carboxymethycellulose, hydroxypropyl methylcellulose, hydroxypropyl cellulose, hydroxyethylcellulose, methylhydroxyethylcellulose, ethylcellulose which may optionally be cross-linked, and sodium carboxymethyl cellulose; vinyls, such as polyvinyl pyrrolidione; glycols, such as polyethylene glycols; acrylics, such as dimethylaminoethyl methacrylate-methacrylate acid ester copolymer, and ethylacrylate-methylmethacrylate copolymer; and other carbohydrate polymers, such as maltodextrins, and polydextrose. Preferably, the inert coating agent contains a hydrophilic film-forming agent, such as hydroxypropyl methylcellulose, so that absorption in vivo is not significantly delayed.

Once the particles or granulated formulations of norastemizole are coated with the inert coating agent, the coated norastemizole may be formulated using standard techniques, including, but not limited to, blending, granulation, compression, or combinations thereof, with other inert and/or reactive excipients, such as lactose, to make various dosage forms, such as tablets, caplets, capsules, troches, and the like.

The preferred amount of norastemizole in all the dosage forms made in accordance with the present invention should be a therapeutically effective amount thereof, which is also a medically acceptable amount thereof. Actual dosage levels of norastemizole in the pharmaceutical compositions of the present invention may be varied so as to obtain an amount of norastemizole which is effective to achieve the desired therapeutic response for a particular patient, pharmaceutical composition of norastemizole, and mode of administration, without being toxic to the patient.

The selected dosage level and frequency of administration of the pharmaceutical compositions of the invention will depend upon a variety of factors including the route of administration, the time of administration, the rate of excretion of the therapeutic agent(s) including norastemizole, the duration of the treatment, other drugs, compounds and/or materials used in combination with norastemizole, the age, sex, weight, condition, general health and prior medical history of the patient being treated, and like factors well known in the medical arts. For example, the dosage regimen is likely to vary with pregnant women, nursing mothers and children relative to healthy adults.

A physician having ordinary skill in the art can readily determine and prescribe the therapeutically effective amount of the pharmaceutical composition required. For example, the physician could start doses of norastemizole employed in the pharmaceutical composition of the present invention at levels lower than that required to achieve the desired therapeutic effect and gradually increase the dosage until the desired effect is achieved.

A suitable daily dose of norastemizole will be that amount of norastemizole which is the lowest effective dose to produce a desired therapeutic effect. Such a therapeutically effective dose will generally depend upon the factors described above. For example, the unit dose of lactose-free norastemizole may contain from about 1 mg to about 200 mg and preferably about 2 mg to about 100 mg. For example, unit dosages may be formulated with 2.5 mg, 5 mg, 10 mg, 12.5 mg, 15 mg, 20 mg, 25 mg, 30 mg, 35 mg, 40 mg, 45 mg, 50 mg, or 62.5 mg of norastemizole. If desired, the effective daily dose of norastemizole may be administered separately at appropriate intervals throughout the day, optionally, in unit dosage forms as two, three, four, five, six or more sub-doses. As previously noted, the preferred dosage forms are tablets, caplets, troches, pastilles, pills, lozenges, syrups, capsules and the like. However, other pharmaceutically acceptable dosage forms such as powders, granules, dragees and the like may be used.

It is noted that all components comprising the dosage forms of norastemizole made in accordance with the present invention preferably meet or exceed the standards for pharmaceutical ingredients and combinations thereof in the USP/NF. The purpose of the USP/NF is to provide authoritative standards and specifications for materials and substances and their preparations that are used in the practice of the healing arts. The USP/NF establish titles, definitions, descriptions, and standards for identity, quality, strength, purity, packaging and labeling, and also, where practicable provide bioavailability, stability, procedures for proper handling and storage and methods for their examination and formulas for their manufacture or preparation.

The lactose-free, non-hygroscopic, anhydrous, large particle, and coated dosage forms of norastemizole described herein and claimed meet the pharmaceutical standards set forth in the USP/NF (e.g., USP XXI/NF XVI) for each of the ingredients as well as the lactose-free, non-hygroscopic, anhydrous, large particle, or coated norastemizole dosage forms made with such ingredients. In effect, the lactose-free, non-hygroscopic, anhydrous, large particle, or coated dosage forms of norastemizole are said to be pharmaceutically acceptable dosage forms made of pharmaceutically acceptable ingredients in pharmaceutically acceptable combinations and pharmaceutically acceptable amounts to at least meet the standards set forth in the USP XXI/NF XVI, incorporated herein in its entirety by reference thereto. In addition, it should be noted that norastemizole can be made according to methods known in the art, including those disclosed in copending U.S. application Ser. No. 08/182,685, filed Jan. 18, 1994, which is incorporated herein by reference thereto for the express purpose of teaching methods to prepare norastemizole.

Stability of a pharmaceutical product may be defined as the capability of a particular formulation, in a specific container, to remain within its physical, chemical, microbiological, therapeutic and toxicological specification, although there are exceptions, and to maintain at least about 90% of labeled potency level. Thus, for example, expiration dating is defined as the time in which the pharmaceutical product will remain stable when stored under recommended conditions.

Many factors affect the stability of a pharmaceutical product, including the stability of the therapeutic ingredient(s), the potential interaction between therapeutic and inactive ingredient(s) (e.g., norastemizole and excipients) and the like. Physical factors such as heat, light and moisture may initiate or accelerate chemical reactions.

For convenience, certain terms employed herein are defined as follows. The term “carrier” as used herein is synonymous with the term “vehicle.” The term “lactose-free” as used herein is intended to mean that the amount of lactose present, if any, in the dosage form of norastemizole is insufficient to cause the incompatibility between norastemizole and lactose discovered by the inventors to detrimentally affect the potency of the norastemizole below about 90% of initial potency over the shelf life of the dosage form. The term “unbound water” as used herein means water that is not present in the form of a stable hydrate of one or more components of the pharmaceutical composition, e.g., alpha lactose monohydrate. Similarly, the term “anhydrous” as used herein means the amount of unbound water present, if any, in the dosage form of norastemizole is insufficient to initiate and/or accelerate the incompatibility between norastemizole and lactose. Further, “anhydrous,” “anhydrous conditions” or “anhydrous nature” as used herein means substantially free of unbound water including moisture. The term “non-hygroscopic” as used herein means the overall formulation is substantially non-hygroscopic, i.e., does not provide unbound water sufficient to initiate and/or accelerate the incompatibility between norastemizole and reactive excipients, such as lactose. The term “additives” is synonymous with the term “excipients” as used herein. The term “substantially free” means less than about 5 weight percent, preferably less than about 1 weight percent, and more preferably less than about 0.1 weight percent. The term “large particle” as used herein means a composition wherein the norastemizole includes about 40 weight percent or more of particles of norastemizole, or a pharmaceutically acceptable salt thereof, having a size of 200 μm or larger, preferably greater than about 250 μm. The terms “coated,” “inert coating,” or “inertly coated” as used herein preferably means an inert coating agent used to coat norastemizole particles and inhibit the interaction of the particles with reactive excipients, such as lactose. Although non-inert coatings suitable for use in conventional pharmaceutical applications are also suitable for use with the lactose-free, non-hygroscopic, anhydrous, and large particle formulations of the invention, it is preferred that any coating used be inert and inhibit the interaction of norastemizole with any reactive excipients.

The term “pharmaceutically acceptable” is used herein to refer to those compounds, materials, compositions and/or dosage forms which are, within the scope of sound medical judgment, suitable for administration to and for use in contact with the tissues and fluids of human beings and animals without excessive toxicity, irritation, allergic response, or other problem or complication, commensurate with a reasonable medically sound benefit/risk ratio.

Further, the term “pharmaceutically acceptable” excipient is employed to mean that there are no untoward chemical or physical incompatibilities between norastemizole or a salt thereof) and any of the excipient components of a given dosage form. For example, an untoward chemical reaction is one wherein the potency of the norastemizole (or salt thereof) is detrimentally reduced or increased due to the addition of one or more excipients. Another example of an untoward chemical reaction is one wherein the taste of the norastemizole (or salt thereof) dosage form becomes excessively sweet, sour or the like to the extent that the dosage form becomes unpalatable. Each excipient must be “acceptable” in the sense of being compatible with the other ingredients of the norastemizole formulation and not injurious to the patient.

Physical incompatibility refers to incompatibility among the various components of the dosage form such as norastemizole (or salt thereof) and any of the excipient(s) thereof. For example, the combination of the excipient(s) and norastemizole may form an excessively hygroscopic mixture or an excessively segregated mixture to the degree that the desired shape of the dosage form (e.g., tablet, troche etc.), its stability or the like cannot be sufficiently maintained to be able to administer the dosage form in compliance with a prescribed dosage regimen as desired.

Most often, antihistamines, such as astemizole or norastemizole, are administered orally by means of solid dosage forms such as tablets, capsules, troches, caplets and the like. Further, capsule dosage forms such as hard gelatin capsules, soft gelatin capsules and the like may also be used. However, tablets remain a preferred dosage form because of the advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives.

In order for medicinal substances or therapeutic ingredients of the present invention (i.e., lactose-free, non-hygroscopic, anhydrous, large particle, or coated norastemizole dosage forms), with or without diluents, to be made into solid dosage forms (e.g., tablets) with pressure, using available equipment, it is necessary that the material, either in crystalline or powdered form, possess a number of physical characteristics. These characteristics include, for example, the ability to flow freely, as a powder to cohere upon compaction, and to be easily released from tooling. Since most materials have none or only some of these properties, methods of tablet formulation and preparation have been developed to impart these desirable characteristics to the material which is to be compressed into a tablet or similar dosage form.

As noted, in addition to the drug or therapeutic ingredient, tablets and similar dosage forms may contain a number of materials referred to as additives. These additives are classified according to the role they play in the formulation of the dosage form such as a tablet, a caplet, a capsule, a troche or the like. One group of additives include, but are not limited to, binders, diluents (fillers), disintegrants and lubricants. In one embodiment the diluent, binder, disintegrant, and lubricant are not the same.

While the discussion below of various additives for use in the present invention specifically refers to lactose-free dosage forms, the skilled artisan will readily understand that a subset of each category includes additives suitable for use in non-hygroscopic, anhydrous, large particle, or coated pharmaceutical compositions of the present invention. In addition, the non-hygroscopic, anhydrous, large particle, or coated pharmaceutical compositions of the present invention may also include lactose or other mono- or di-saccharides as excipients. In another embodiment, inorganic bisulfites may be used to improve the stability of any of the norastemizole compositions herein.

For non-hygroscopic formulations, special precautions must be exercised in choosing excipients and additives, such that overall, there is no propensity for moisture sorption (absorption or adsorption) in the absence of suitable environmental controls. For example, excipients for use in such formulations include, but are not limited to, alpha lactose monohydrate, mannitol and the like.

For anhydrous formulations, suitable anhydrous or low moisture forms of the below identified excipients or additives should be used, for example, AVICEL-PH-103™ and Starch 1500 LM.

A binder is used to provide a free-flowing powder from the mix of tablet ingredients so that the material will flow when used on a tablet machine. The binder also provides a cohesiveness to the norastemizole tablet. Too little binder will give flow problems and yield tablets that do not maintain their integrity. Too much may adversely affect the release (dissolution rate) of the drug from the tablet. Thus, a sufficient amount of binder should be incorporated into the tablet to provide a free-flowing mix of the tablet ingredients without adversely affecting the dissolution rate of the drug ingredients from the tablet. With lower dose tablets, the need for good compressibility can be eliminated to a certain extent by the use of suitable diluting excipients called compression aids. The amount of binder used varies upon the type of formulation and mode of administration, and is readily discernible to those of ordinary skill in the art.

Binders suitable for use with the lactose-free, non-hygroscopic, anhydrous, large particle, or coated dosage formulations of norastemizole made in accordance with the present invention include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose or mixtures thereof.

Suitable forms of microcrystalline cellulose are, for example, the materials sold as AVICEL-PH-101, AVICEL-PH-103 and AVICEL-PH-105 (available from FMC Corporation, American Viscose Division, Avicel Sales, Marcus Hook, Pa., U.S.A.). An exemplary suitable binder is a mixture of microcrystalline cellulose and sodium carboxymethyl cellulose sold as AVICEL RC-581 by FMC Corporation.

Most commercial tablets weigh from about 100 mg to about 500 mg. Thus, for many potent drugs including dosage forms of norastemizole, a filler comprises a large portion of the tablet. Fillers (e.g., diluents) are used to give the powder (e.g., in the tablet or capsule) bulk so that an acceptable size tablet, capsule or other desirable dosage form is produced. Typically, therapeutic ingredients are formed in a convenient dosage form of suitable size by the incorporation of a diluent therewith. As with the binder, binding of the drug to the filler may occur and affect bioavailability. Consequently, a sufficient amount of filler should be used to achieve a desired dilution ratio without detrimentally affecting release of the drug ingredient(s) from the dosage form containing the filler. Further, a filler that is physically and chemically compatible with the therapeutic ingredient(s) of the dosage form should be used. Thus, as noted, lactose should not be used with norastemizole to form the dosage forms of norastemizole made in accordance with the present invention if precautions have not been taken to eliminate unbound water. It is also preferable that the lactose-free dosage forms of norastemizole according to the present invention do not include mono- or di-saccharides, such as, but not limited to, glucose, sucrose and fructose. The amount of filler used varies upon the type of formulation and mode of administration, and is readily discernible to those of ordinary skill in the art.

Examples of suitable fillers for use with the lactose-free dosage forms of norastemizole made in accordance with the present invention include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, or mixtures thereof.

The binder/filler in pharmaceutical compositions of the present invention is typically present in about 15 to 99 weight percent, more specifically about 50 to about 99 weight percent of the pharmaceutical composition.

Disintegrants are used to cause the tablet to disintegrate when exposed to an aqueous environment. Too much of a disintegrant will produce tablets which may disintegrate in the bottle due to atmospheric moisture and provide unbound water sufficient to initiate and/or accelerate norastemizole lactose interaction. Too little may be insufficient for disintegration to occur and may thus alter the rate and extent of release of the drug ingredient(s) from the dosage form. Thus, a sufficient amount of disintegrant that is neither too little nor too much to detrimentally alter the release of the drug ingredient(s) should be used to form the dosage forms of norastemizole made according to the present invention. The amount of disintegrant used varies based upon the type of formulation and mode of administration, and is readily discernible to those of ordinary skill in the art. Typically, about 0.5 to about 15 weight percent of the pharmaceutical composition is disintegrant, preferably about 1 to about 5 weight percent of the pharmaceutical composition is disintegrant.

Suitable disintegrants that may be used to form the lactose-free dosage forms of norastemizole made in accordance with the present invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Based on the physicochemical properties of norastemizole, it is typically desirable to formulate the lactose-free, non-hygroscopic, anhydrous, large particle, or coated pharmaceutical compositions of norastemizole such that they dissolve fairly rapidly upon administration to the subject, e.g., in the subject's stomach. Thus, in a preferred embodiment, the lactose-free, non-hygroscopic, anhydrous, large particle, or coated pharmaceutical compositions of the present invention include a super disintegrant, such as, but not limited to, croscarmellose sodium or sodium starch glycolate. The term “super disintegrant,” as used herein, means a disintegrant that results in rapid disintegration of the norastemizole in the stomach after oral administration. This is particularly important since the maximal solubility of norstemizole is in acidic media. Accordingly, it is desirable to have orally administered dosage forms of norastemizole rapidly disintegrate in the acidic environment of the stomach to facilitate the rapid absorption of norastemizole which may result in a more rapid onset of action. Croscarmellose sodium is a particularly advantageous super disintegrant. Croscarmellose sodium is more effective than ordinary disintegrants based on its capacity to rapidly swell to many times its original volume when exposed to water. This swelling quickly disperses the other formulation components, including the norastemizole.

Whatever the dose, adhesion of the dosage form ingredients to the punches of the tableting machine must be avoided. For example, when drug (e.g., norastemizole) accumulates on the punch surfaces, it causes the tablet surface to become pitted and therefore unacceptable. Also, sticking of drug or other dosage form ingredients in this way requires unnecessarily high ejection forces when removing the tablet from the die. Excessive ejection forces may lead to a high breakage rate and increase the cost of production not to mention excessive wear and tear on the dies. In practice, it is possible to reduce sticking by wet-massing or by the use of high levels of lubricants, e.g., magnesium stearate. However, selection of a drug salt with good anti-adhesion properties also minimizes these problems.

As noted, the lubricant is used to enhance the flow of the lactose-free norastemizole tableting powder mix to the tablet machine and to prevent sticking of the tablet in the die after the tablet is compressed. Too little lubricant will not permit satisfactory tablets to be made and too much may produce a tablet with a water-impervious hydrophobic coating. Because lubricants are usually hydrophobic materials such as stearic acid, magnesium stearate, calcium stearate and the like, a water-impervious hydrophobic coating may be formed by the use of too much lubricant. Further, a water-impervious hydrophobic coating can inhibit disintegration of the tablet and dissolution of the drug ingredient(s). Thus, a sufficient amount of lubricant should be used that readily allows release of the compressed tablet from the die without forming a water-impervious hydrophobic coating that detrimentally interferes with the desired disintegration and/or dissolution of the drug ingredient(s).

Suitable lubricants for use with the lactose-free dosage forms of norastemizole made in accordance with the present invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, or mixtures thereof. Additional lubricants include, for example, a syloid silica gel (AEROSIL 200, manufactured by W.R. Grace Co. of Baltimore Md.), a coagulated aerosol of synthetic silica (marketed by Deaussa Co. of Plano, Tex.), CAB-O-SIL (a pyrogenic silicon dioxide product sold by Cabot Co. of Boston, Mass.) or mixtures thereof. A lubricant may optionally be added, typically in an amount of less than about 1 weight percent of the pharmaceutical composition.

As noted above, preferred norastemizole dosage forms made in accordance with the present invention comprise norastemizole, microcrystalline cellulose, pre-gelatinized starch, and magnesium stearate. Preferably, norastemizole is present in an amount of from about 1 to 50 percent, the microcrystalline cellulose is present in an amount of from about 20 to 90 percent, the pregelatanized starch is present in an amount of from about 5 to 75 percent, the croscarmellose sodium is present in an amount of from about 1 to 5 percent, and the magnesium stearate is present in an amount of from about 0.05 to 0.8 percent by weight of the pharmaceutical composition.

Another class of additives for use with the dosage forms of norastemizole include, but are not limited to, anti-caking agents, antimicrobial preservatives, coating agents, colorants, desiccants, flavors and perfumes, plasticizers, viscosity increasing agents, sweeteners, buffering agents, humectants and the like.

Suitable anti-caking agents for use with the lactose-free dosage forms of norastemizole made in accordance with the present invention include, but are not limited to, calcium silicate, magnesium silicate, silicon dioxide, colloidal silicon dioxide, talc or mixtures thereof.

Suitable antimicrobial preservatives for use with the lactose-free dosage forms of norastemizole made in accordance with the present invention include, but are not limited to, benzalkonium chloride solution, benzethonium chloride, benzoic acid, benzyl alcohol, butyl paraben, cetylpyridinium chloride, chlorobutanol, cresol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymol or mixtures thereof.

Suitable coating agents for use with the lactose-free dosage forms of norastemizole made in accordance with the present invention include, but are not limited to, sodium carboxymethyl cellulose, cellulose acetate phthalate, ethylcellulose, gelatin, pharmaceutical glaze, hydroxypropyl cellulose, hydroxypropyl methylcellulose (e.g., Nos.: 2208, 2906, 2910), hydroxypropyl methyl cellulose phthalate (e.g., Nos.: 200731, 220824), methylcellulose, polyethylene glycol, polyvinyl acetate phthalate, shellac, sucrose, titanium dioxide, carnauba wax, microcrystalline wax or mixtures thereof. The amount of coating agent and the carrier vehicle (aqueous or non-aqueous) used varies upon the type of formulation and mode of administration, and is readily discernible to those of ordinary skill in the art.

A coating of a film forming polymer may optionally be applied to the norastemizole tablet (e.g., a capsule shaped tablet often referred to as a caplet) in accordance with the present invention by using one of several types of equipment such as a conventional coating pan, Accelacota, High-Cola or Worster air suspension column. Such equipment typically has an exhaust-system to remove dust and solvent or water vapors to facilitate quick drying. Spray guns or other suitable atomizing equipment may be introduced into the coating pans to provide spray patterns conducive to rapid and uniform coverage of the tablet bed. Normally, heated or cold drying air is introduced over the tablet bed in a continuous or alternate fashion with a spray cycle to expedite drying of the film coating solution. For non-hygroscopic, anhydrous, large particle, or coated pharmaceutical compositions of the invention containing reactive excipients, such as lactose, non-aqueous operations are preferred, e.g., non-aqueous coating should be used.

The coating solution may be sprayed by using positive pneumatic displacement or peristaltic pump systems in a continuous or intermittent spray-dry cycle. The particular type of spray application is selected depending upon the drying efficiency of the coating pan.

In most cases, the coating material is sprayed until the lactose-free, non-hygroscopic, large particle, anhydrous, or coated norastemizole tablets are uniformly coated to the desired thickness and the desired appearance of the tablet is achieved. Many different types of coatings may be applied such as enteric, slow release coatings or rapidly dissolving type coatings for fast acting tablets. Preferably, rapidly dissolving type coatings are used to permit more rapid release of the active ingredients, resulting in hastened onset. The thickness of the coating of the film forming polymer applied to a tablet, for example, may vary. However, it is preferred that the thickness simulate the appearance, feel (tactile and mouth feel) and function of a gelatin capsule. Where more rapid or delayed release of the therapeutic agent(s) is desired, one skilled in the art would easily recognize the film type and thickness, if any, to use based on characteristics such as desired blood levels of active ingredient, rate of release, solubility of active ingredient, and desired performance of the dosage form.

A number of suitable film forming agents for use in coating a final dosage form, such as tablets comprising the present lactose-free, non-hygroscopic, anhydrous, large particle or coated formulations of norastemizole include, for example, methylcellulose, hydroxypropyl methyl cellulose (PHARMACOAT 606 6 cps), polyvinylpyrrolidone (povidone), ethylcellulose (ETHOCEL 10 cps), various derivatives of methacrylic acids and methacrylic acid esters, cellulose acetate phthalate or mixtures thereof.

Suitable colorants for use with the lactose-free dosage forms of norastemizole made in accordance with the present invention include, but are not limited to, pharmaceutically acceptable dyes and lakes, caramel, red ferric oxide, yellow ferric oxide or mixtures thereof. Suitable desiccants for use with the lactose-free, anhydrous, large particle, or coated norastemizole dosage formulations made in accordance with the present invention include, but are not limited to, calcium chloride, calcium sulfate, silica gel or mixtures thereof.

Suitable flavors for use with the lactose-free dosage forms of norastemizole made in accordance with the present invention include, but are not limited to, acacia, tragacanth, almond oil, anethole, anise oil, benzaldehyde, caraway, caraway oil, cardamom oil, cardamom seed, compound cardamom tincture, cherry juice, cinnamon, cinnamon oil, clove oil, cocoa, coriander oil, eriodictyon, eriodictyon fluidextract, ethyl acetate, ethyl vanillin, eucalyptus oil, fennel oil, glycyrrhiza, pure glycyrrhiza extract, glycyrrhiza fluidextract, lavender oil, lemon oil, menthol, methyl salicylate, monosodium glutamate, nutmeg oil, orange flower oil, orange flower water, orange oil, sweet orange peel tincture, compound orange spirit, peppermint, peppermint oil, peppermint spirit, pine needle oil, rose oil, stronger rose water, spearmint, spearmint oil, thymol, tolu balsam tincture, vanilla, vanilla tincture, and vanillin or mixture thereof.

Suitable plasticizers for use with the lactose-free dosage forms of norastemizole made in accordance with the present invention include, but are not limited to, castor oil, diacetylated monoglycerides, diethyl phthalate, glycerin, mono-and di-acetylated monoglycerides, polyethylene glycol, propylene glycol, and triacetin or mixtures thereof.

Suitable viscosity increasing agents for use with the lactose-free dosage forms of norastemizole made in accordance with the present invention include, but are not limited to, acacia, agar, alamic acid, aluminum monostearate, bentonite, bentonite magma, carbomer 934, carboxymethylcellulose calcium, carboxymethylcellulose sodium, carboxymethylcellulose sodium 12, carrageenan, cellulose, microcrystalline cellulose, gelatin, guar gum, hydroxyethyl cellulose, hydroxypropyl cellulose, hydroxypropyl methylcellulose (Nos. 2208; 2906; 2910), magnesium aluminum silicate, methylcellulose, pectin, polyvinyl alcohol, povidone, silica gel, colloidal silicon dioxide, sodium alginate, tragacanth and xanthan gum or mixtures thereof.

Suitable sweetening agents for use with the lactose-free dosage forms of norastemizole made in accordance with the present invention include, but are not limited to, aspartame, dextrates, mannitol, saccharin, saccharin calcium, saccharin sodium, sorbitol, sorbitol solution, or mixtures thereof.

Suitable buffering agents for use with the lactose-free dosage forms of norastemizole made in accordance with the present invention include, but are not limited to, magnesium hydroxide, aluminum hydroxide and the like, or mixtures thereof. Suitable humectants include, but are not limited to, glycerol, other humectants or mixtures thereof. The dosage forms of norastemizole of the present invention may further include one or more of the following: (1) dissolution retarding agents, such as paraffin; (2) absorption accelerators, such as quaternary ammonium compounds; (3) wetting agents, such as, for example, cetyl alcohol and glycerol monostearate; (4) absorbents, such as kaolin and bentonite clay; (5) antioxidants, such as water soluble antioxidants (e.g., ascorbic acid, cysteine hydrochloride, sodium bisulfate, sodium metabisulfate, sodium sulfite and the like), oil soluble antioxidants (e.g., ascorbyl palmitate, hydroxyanisole (BHA), butylated hydroxy toluene (BHT), lecithin, propyl gallate, alpha-tocopherol and the like); and (6) metal chelating agents, such as citric acid, ethylenediamine tetracetic acid (EDTA), sorbitol, tartaric acid, phosphoric acid and the like.

The lactose-free, non-hygroscopic, anhydrous, large particle, or coated norastemizole dosage forms of the present invention may also be provided in the form of hard or soft capsules, for example, of gelatin or other suitable materials together with various excipients previously noted with regard to tablets. For the formation of tablets, the norastemizole is combined with one or more excipients (e.g., diluents, binders, disintegrants, dispersing agents, surface-active agents, lubricants, coating materials, flavoring agents, coloring agents, solvents, viscosity increasing agents, suspending agents, sweeteners, colorants, dyes and the like) in various proportions using traditional tableting equipment such as twin shell or “v” blenders by known procedures to manufacture chemically and thermally stable dosage forms (e.g., tablets, caplets and the like) containing a uniform distribution and blending of therapeutic agents. The exact amounts of each of the various excipients may be readily determined by those of ordinary skill in the pharmaceutical art.

Large-scale production of lactose-free, non-hygroscopic, anhydrous, large particle, or coated dosage forms of norastemizole made in accordance with the present invention may require, in addition to the therapeutic drug ingredient(s), additives including, but not limited to, diluents, binders, lubricants, disintegrants, colorants, flavors, sweetening agents and the like or mixtures thereof. By the incorporation of these and other additives, a variety of dosage forms (e.g., tablets, capsules, caplets, troches and the like) may be made. These include, for example, hard gelatin capsules, caplets, sugar-coated tablets, enteric-coated tablets to delay action, multiple compressed tablets, prolonged-action tablets, tablets for solution, effervescent tablets, buccal and sublingual tablets, troches and the like. Sugar-coating preferably does not include lactose or mono- or di-saccharides, except in norastemizole formulations substantially free of unbound water.

Tablets of the lactose-free, non-hygroscopic, anhydrous, large particle, or coated dosage forms of norastemizole of the present invention are typically made by molding, by compression or by generally accepted tablet forming methods. Accordingly, compressed tablets are usually prepared by large-scale production methods while molded tablets often involve small-scale operations. For example, there are three general methods of tablet preparation for making the dosage forms of norastemizole: (1) the wet-granulation method; (2) the dry-granulation method; and (3) direct compression. These methods are well known to those skilled in the art. See Remington's Pharmaceutical Sciences, 16th and 18th Eds., Mack Publishing Co., Easton, Pa. (1980 and 1990). See also U.S. Pharmacopeia XXI, U.S. Pharmacopeial Convention, Inc., Rockville, Md. (1985). Preferably for non-hygroscopic or anhydrous dosage forms, wet granulation is not used.

Various tablet formulations of the lactose-free, non-hygroscopic, anhydrous, large particle, or coated dosage forms of norastemizole may be made in accordance with the present invention. These include tablet dosage forms such as sugar-coated tablets, film-coated tablets, enteric-coated tablets, multiple-compressed tablets, prolonged action tablets and the like. Lactose-free, non-hygroscopic, anhydrous, large particle, or inert coated norastemizole sugar-coated tablets (SCT) are compressed tablets containing a sugar coating. Such coatings may be colored and are beneficial in covering up drug substances possessing objectionable tastes or odors and in protecting materials sensitive to oxidation. Lactose-free, non-hygroscopic, anhydrous, large particle, or coated norastemizole film-coated tablets (FCT) are compressed tablets which are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Lactose-free, non-hygroscopic, anhydrous, large particle or coated norastemizole enteric-coated tablets (ECT) are compressed tablets coated with substances that resist dissolution in gastric fluid but disintegrate in the intestine. Enteric coating can be used for tablets containing drug substances which are inactivated or destroyed in the stomach, for those which irritate the mucosa or as a means of delayed release of the medication.

Lactose-free, non-hygroscopic, anhydrous, large particle, or coated norastemizole multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These lactose-free, non-hygroscopic, or anhydrous norastemizole tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of norastemizole tablets (i.e., layered tablets and press-coated tablets) may be used, for example, in the design of prolonged-action dosage forms.

Lactose-free, non-hygroscopic, anhydrous, large particle, or coated norastemizole prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

The method of preparation and the additives to be incorporated into a lactose-free, non-hygroscopic, anhydrous, large particle, or coated norastemizole tablet are selected in order to give the tablet formulation the desirable physical characteristics while allowing the rapid compression of tablets. After compression, the tablets preferably should have a number of additional attributes such as appearance, hardness, disintegration ability and uniformity which are influenced both by the method of preparation and by the additives present in the tablet formulation.

The basic unit in all tablet compression equipment includes a lower punch which fits into a die from the bottom and an upper punch, having a head of generally the same shape and dimensions as that of the lower punch, which enters the die cavity from the top after the tableting material fills the die cavity. The tablet is formed by pressure applied on the punches. Subsequently, the tablet is ejected from the die. The weight of the tablet is determined by the volume of the material which fills the die cavity.

The ability of the lactose-free, non-hygroscopic, anhydrous, large particle, or coated norastemizole tablet or dosage form granulation to flow freely into the die cavity is important in insuring an uniform fill. The flowability of the granulation is also important to insure continuous movement of the granulation from the source of supply or feed hopper. Further, if the tablet granulation does not possess cohesive properties, after compression the tablet will crumble and fall apart on handling. Even further, as the punches must move freely within the die and the tablet must be readily ejected from the punch faces, the tableting material must have a degree of lubrication to minimize friction and to allow for the removal of the compressed tablet. A granulating agent may be added to facilitate granulation. The amount of granulating agent used varies upon the type of formulation and mode of administration, and is readily discernible to those of ordinary skill in the art. Typically, about 5 to about 15 weight percent of granulating agent is used in the pharmaceutical formulation. Preferably, when lactose is present in the anhydrous or non-hygroscopic compositions of the present invention, the granulating agent should be non-aqueous.

Further, it is noted that stable lactose-free, non-hygroscopic, anhydrous, large particle, or coated norastemizole tablets or other dosage forms thereof retain their original size, shape, weight and color under normal handling and storage conditions throughout their shelf life. Thus, for example, excessive powder or solid particles at the bottom of the container, cracks or chips on the face of a tablet, or appearance of crystals on the surface of tablets or on container walls are indicative of physical instability of uncoated tablets. Hence, the effect of mild, uniform and reproducible shaking and tumbling of tablets should be undertaken to insure that the tablets have sufficient physical stability. Tablet hardness can be determined by commercially available hardness testers. In addition, the in vitro availability of the active ingredient should not change appreciably with time.

The lactose-free pharmaceutical compositions of the present invention may also be formulated in a soft elastic gelatin capsule unit dosage form by using conventional methods, well-known in the art (see, e.g., Ebert, Pharm. Tech., 1(5):44-50 (1977)). Soft elastic gelatin capsules have a soft, globular, gelatin shell somewhat thicker than that of hard gelatin capsules, wherein a gelatin is plasticized by the addition of glycerin, sorbitol, or a similar polyol. The hardness of the capsule shell may be changed by varying the type of gelatin and the amounts of plasticizer and water. The soft gelatin shells may contain a preservative (such as methyl-and propylparabens and sorbic acid) to prevent the growth of fungi. The active ingredient may be dissolved or suspended in a liquid vehicle or carrier, such as vegetable or mineral oils, glycols such as polyethylene glycol and propylene glycol, triglycerides, surfactants such as polysorbates, or a combination thereof.

The tablets, and other dosage forms of the pharmaceutical compositions of the present invention, such as dragees, capsules, pills and granules, may optionally be scored or prepared with coatings and shells, such as enteric coatings and other coatings well known in the pharmaceutical formulating art.

The pharmaceutical compositions of the present invention may also be formulated so as to provide slow or controlled release of the active ingredient therein using, for example, hydroxypropylmethyl cellulose in varying proportions to provide the desired release profile, other polymer matrices, liposomes and/or microspheres.

The pharmaceutical compositions of the present invention may also be formulated so as to provide a solid quick dissolving dosage form, i.e., a dosage form that rapidly dissolves in the mouth when placed on the tongue. preferably, the quick dissolving dosage form is in the form of a tablet. Quick dissolving dosage forms are manufactured using excipients that have a high water solubility and by compressing their components at much lower pressures than typically used to manufacture solid dosage forms. Often, quick dissolving dosage forms include a sweetener to provide a sweet taste when the dosage form dissolves on the tongue. Preferably, the norastemizole in these formulations is coated to avoid the bitter taste associated with norastemizole.

Unless indicated otherwise, all percentages noted herein are percentages by weight based on the total weight of all the components of a particular dosage form.

The lactose-free, non-hygroscopic, anhydrous, large particle, or coated norastemizole compositions of the present invention may further contain, for example, an analgesic, a decongestant, a cough suppressant, or an expectorant. In a preferred embodiment the pharmaceutical composition comprises pseudoephedrine, or a pharmaceutically acceptable salt thereof.

The norastemizole compositions of the present invention may also be formulated as a liquid oral dosage form (e.g., a solution, suspension, or elixir). Liquid oral dosage forms are prepared by combining the active ingredient in a suitable solvent to form a solution, suspension, syrup, or elixir of the active ingredient in the liquid.

As used herein, the term “elixir” means a solution of norastemizole in a solvent containing water and alcohol.

As used herein, the term “syrup” means a concentrated solution of sugar, such as sucrose, in water or other aqueous liquid, optionally containing polyols, such as glycerin or sorbitol to retard crystallization of the sugar or increase solubility of the added ingredients.

The solutions, suspensions, syrups, and elixirs may optionally comprise other additives including, but not limited to, glycerin, sorbitol, propylene glycol, sugars, flavoring agents, buffers, and stabilizers.

The incompatibility of norastemizole with lactose is illustrated in Table I below. The effect of lactose on norastemizole at various temperatures (e.g., 25° C., 40° C. and 60° C.), at various relative humidity levels (e.g., 60% and 75% relative humidity) and at various times (e.g., zero, 1 week, 1 month, 2 months, 3 months, 6 months and 9 months) was evaluated. The results of such evaluation are presented in Table I. The level of impurities within the capsules tested was measured using high pressure liquid chromatography (HPLC), and is presented in Table I as a percentage of the dosage form tested. Note that discoloration from the initial white opaque appearance is an indication of incompatibility between norastemizole and lactose, which is corroborated by increased percentages of impurities detected by HPLC.

TABLE I
STABILITY OF NORASTEMIZOLE CAPSULES

| Tem- | Relative | | | HPLC | HPLC |
|---|---|---|---|---|---|
| perature | Humidity | | | Impurities | Assay |
| (° C.) | (%) | Time | Appearance | (%) | (%) |
| 25 | 60 | 0 | white opaque | 0.10 | 101.0 |
| | | | capsules | | |
| 25 | 60 | 1 week | white opaque | 0.12 | 97.1 |
| | | | capsules | | |
| 25 | 60 | 1 month | white opaque | 0.25 | 99.3 |
| | | | capsules | | |
| 25 | 60 | 2 months | white opaque | 0.39 | 95.7 |
| | | | capsules | | |
| 25 | 60 | 3 months | white opaque | 0.35 | 99.9 |
| | | | capsules | | |
| 25 | 60 | 6 months | yellow/white | 1.53 | 96.8 |
| | | | opaque capsules | | |
| 25 | 60 | 9 months | off white | 1.63 | 94.7 |
| | | | opaque capsules | | |
| 40 | 75 | 1 week | white opaque | 0.46 | 98.5 |
| | | | capsules | | |
| 40 | 75 | 1 month | white opaque | 1.45 | 98.9 |
| | | | capsules | | |
| 40 | 75 | 2 months | white opaque | 3.42 | 95.0 |
| | | | capsules | | |
| 40 | 75 | 3 months | slightly dark | 7.40 | 88.2 |
| | | | off-white capsules | | |
| 40 | 75 | 6 months | beige opaque | 18.80 | 78.5 |
| | | | capsules | | |
| 40 | 75 | 9 months | beige opaque | 22.92 | 70.7 |
| | | | capsules | | |
| 60 | 75 | 1 week | white opaque | 13.90 | 83.0 |
| | | | capsules | | |
| 60 | 75 | 1 month | white opaque | 36.87 | not |
| | | | capsules | | done |

*Stability of norastemizole when filled into hard gelatin capsules with lactose, approximately 25 mg norastemizole and 4.975 g of lactose; even at 25° C., capsules showed increased impurities (HPLC Impurities) and reduced in vitro potency (HPLC Assay) after 6 months and 9 months indicating incompatibility between lactose and norastemizole.

Thus, the results show that when norastemizole was formulated with lactose, filled into hard gelatin capsules, and stored in a non-hermetically sealed container, the formulation was not chemically stable at elevated temperatures and humidity. Moreover, even at 25° C. with 60% relative humidity, the norastemizole/lactose capsules showed increased impurities and reduced in vitro potency after six (6) and nine (9) months, indicating incompatibility between norastemizole and lactose.

In an effort to identify excipients, other than lactose, suitable for use with norastemizole, an excipient study was performed using a variety of classes of other excipients. Examples of excipients tested include corn starch, calcium sulfate dihydrate, calcium stearate, sucrose, fructose, calcium carbonate, microcrystalline cellulose, maltodextrin, CaHPO₄●2H₂O, CaHPO₄, magnesium stearate, starch 1500®, croscarmellose sodium or mixtures thereof.

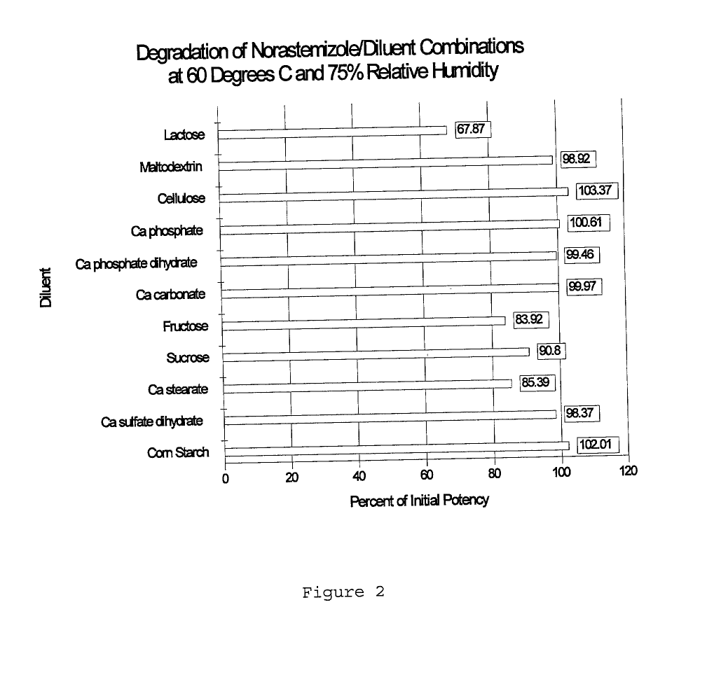

The effect of various excipients on the degradation of norastemizole is depicted in FIG. 2, wherein the norastemizole/excipient combination was exposed to a temperature of 60° C. and relative humidity of 75%, and stored in a non-hermetically sealed container, which are typical conditions for excipient compatibility studies.

As is clear from FIG. 2, norastemizole and lactose are clearly incompatible because of the sharp drop in the potency of the drug. However, no such drastic drop in potency is seen between norastemizole and the other excipients tested. However, the daily dosage may need to be adjusted to take into account the potency variations among the excipients seen and illustrated in FIG. 2.

FIG. 2 also provides some indication that mono- and di-saccharide excipients should also preferably be avoided in norastemizole formulations, e.g., as shown by the degradation observed with norastemizole/sucrose combinations.

The above results were obtained using screw-cap containers, high temperature and humidity which is a widely-accepted means for determining the interactions of compounds with excipients under accelerated conditions. Applicants have also found that norastemizole alone, when stored under high humidity conditions (thus exposed to significant unbound water), is extremely stable over long periods of time. Another study was conducted to assess the effects of moisture changes on the lactose/norastemizole interactions.

In 20-ml amber glass crimp-top vials, the following samples were prepared:

Norastemizole Neat

Norastemizole 20%/Lactose 80%

Norastemizole 20%/Lactose 80% with H₂O 5%

Norastemizole 1%/Lactose 99%

Norastemizole 1%/Lactose 99% with H₂O 5%

The crimped vials were held at 60° C. for 14 days and assayed for norastemizole.

The results show that the incompatibility of norastemizole and lactose is greatly reduced when unbound water is not present and the container is hermetically sealed. Indeed, the moisture effect on the reaction rate is significant. Where unbound water was not purposely added to the well sealed containers, the differences were not substantially different than the control, i.e., neat norastemizole. Reduced potency in the presence of unbound water was observed, whereas reductions in potency comparable to lactose-free or neat norastemizole were observed in the absence of unbound water.

| | % Norastemizole |
|---|---|
| | (Assay Results) |
| Norastemizole Neat | 96.90 |
| Norastemizole 20%/Lactose 80% | 98.33 |
| Norastemizole 20%/Lactose 80% with H₂O 5% | 65.16 |
| Norastemizole 1%/Lactose 99% | 92.59 |
| Norastemizole 1%/Lactose 99% with H₂O 5% | 77.22 |

A lactose-free, non-hygroscopic, anhydrous, large particle, or coated norastemizole dosage formulation such as a troche, a tablet or a capsule may be formed by combining norastemizole, or a pharmaceutically acceptable salt thereof, with one or more pharmaceutically compatible excipients, as described above, in pharmaceutically compatible amounts to yield a unit dose norastemizole dosage formulation containing from about 1 mg to about 200 mg of norastemizole, and preferably containing from about 2 mg to about 100 mg of norastemizole. The tablet, troche or capsule dosage formulation may be formed, for example, by methods well known in the art including wet granulation, dry granulation or compression molding. Again, wet granulation is not useful for non-hygroscopic or anhydrous formulations. Other methods for forming tablets, troches and capsules, well known in the art, may be used. However, compression molding is preferred for the formulation of tablets and troches. For capsules, hard gelatin capsule shells are preferred which are filled with norastemizole and one or more excipients.

STARCH 1500® is a pre-gelatinized starch manufactured by Colorcon Ltd. that is not recommended for use in amounts exceeding 75 weight percent. In addition, when magnesium stearate is used as a lubricant with STARCH 1500®, amounts greater than 0.25 weight percent of magnesium stearate should not be used, as this may have an adverse effect on dissolution. This adverse effect on dissolution in formulations of STARCH 1500® and greater than 0.25 weight percent of magnesium stearate is particularly important for compounds having relatively low water-solubility, such as norastemizole.

Having described the invention, the following examples illustrate preferred embodiments in accordance with the presently claimed invention. It is understood that the examples are illustrative and do not limit the scope or breadth of the appended claims.

### EXAMPLE 1

### Hard Gelatin Capsule Unit Dosage Forms (Lactose-Free)

| | 2.5 mg | | |
|---|---|---|---|
| | capsule | 5 mg capsule | 20 mg capsule |
| | (amount in | (amount in | (amount in |
| Component | mg) | mg) | mg) |
| Norastemizole | 2.5 | 5.0 | 20.0 |
| Microcrystal- | 90.0 | 90.0 | 90.0 |
| line | | | |
| Cellulose | | | |
| Pre- | 100.3 | 97.8 | 82.8 |
| gelatinized | | | |
| Starch | | | |
| Croscarmellose | 7.0 | 7.0 | 7.0 |
| Magnesium | 0.2 | 0.2 | 0.2 |
| Stearate | | | |

### EXAMPLE 2

### Hard Gelatin Capsule Unit Dosage Forms (Non-Hygroscopic)

| | 2.5 mg | | |
|---|---|---|---|
| | capsule | 5 mg capsule | 20 mg capsule |
| | (amount in | (amount in | (amount in |
| Component | mg) | mg) | mg) |
| Norastemizole | 2.5 | 5.0 | 20.0 |
| α-lactose | 197.3 | 144.8 | 179.8 |
| monohydrate | | | |
| Magnesium | 0.2 | 0.2 | 0.2 |
| Stearate | | | |

### EXAMPLE 3

### Hard Gelatin Capsule Unit Dosage Forms (Anhydrous)

| | 2.5 mg | | |
|---|---|---|---|
| | capsule | 5 mg capsule | 20 mg capsule |
| | (amount in | (amount in | (amount in |
| Component | mg) | mg) | mg) |
| Norastemizole | 2.5 | 5.0 | 20.0 |
| AVICEL-PH-103 | 50.0 | 50.0 | 50.0 |
| Starch 1500 | 97.3 | 94.8 | 79.8 |
| LM | | | |
| α-lactose | 50.0 | 50.0 | 50.0 |
| (anhydrous) | | | |
| Magnesium | 0.2 | 0.2 | 0.2 |
| Stearate | | | |

The active ingredient is sieved and blended with the excipients listed. The mixture is filled into suitably sized two-piece hard gelatin capsules using suitable machinery and methods well known in the art. See Remington's Pharmaceutical Sciences, 16th or 18th Editions, each incorporated herein in its entirety by reference thereto. Other doses may be prepared by altering the fill weight and, if necessary, changing the capsule size to suit. Any of the stable, non-lactose, non-hygroscopic, and anhydrous hard gelatin capsule formulations above may be formed.

### EXAMPLE 4

### Compressed Tablet Formulations (Lactose-Free)

| | 2.5 mg tablet | 5 mg tablet | 20 mg tablet |
|---|---|---|---|
| | (amount in | (amount in | (amount in |
| Component | mg) | mg) | mg) |
| Norastemizole | 2.5 | 5.0 | 20.0 |
| Microcrystal- | 90.0 | 90.0 | 90.0 |
| line | | | |
| Cellulose | | | |
| Pregelatinized | 100.3 | 97.8 | 82.8 |
| Starch | | | |
| Croscarmellose | 7.0 | 7.0 | 7.0 |
| Magnesium | 0.2 | 0.2 | 0.2 |
| Stearate | | | |

The active ingredient is sieved through a suitable sieve and blended with the non-lactose excipients until a uniform blend is formed. The dry blend is screened and blended with the magnesium stearate. The resulting powder blend is then compressed into tablets of desired shape and size. Tablets of other strengths may be prepared by altering the ratio of the active ingredient (i.e., norastemizole) to the excipient(s) or modifying the tablet weight.

### EXAMPLE 5

### Wet Granulation (Lactose-Free)

| | Quantity per Tablet (mg) | | |
|---|---|---|---|
| Component | Formulation A | Formulation B | Formulation C |
| Norastemizole | 25 | 50 | 100 |
| Pre-gelatinized starch | 100-150 | 100-125 | 50-100 |
| Microcrystalline | 0-75 | 0-50 | 0-50 |
| cellulose | | | |
| Povidone | 7.5 | — | 7.5 |
| Polyethylene glycol | — | 10-30 | — |
| Croscarmellose | 10 | — | 10 |
| Sodium starch | — | 5-15 | — |
| glycolate | | | |
| Magnesium stearate | 1.5 | 1.5 | 1.5 |
| FDC Yellow #2 lake | 1.25 | 1.25 | 1.25 |

The active ingredient is sieved through a suitable screen and blended with the non-lactose excipients (excluding half of the croscarmellose (or sodium starch glycolate) and all of the microcrystalline cellulose) until a uniform blend is formed. Suitable volumes of water are added and the powder granulated. After drying, the granules are screened and blended with the microcrystalline cellulose, the remainder of croscarmellose or sodium starch glycolate, and briefly with the magnesium stearate. The resulting free-flowing powder is then compressed into tablets of desired shape and size. Tablets of other strengths may be prepared by altering the ratio of the active ingredient (i.e., norastemizole) to the excipients or modifying the tablet weight.

### EXAMPLE 6

### Direct Compression

Quantity per Tablet (mg)

| | Formulation | Formulation |
|---|---|---|
| Component | A | B |
| Norastemizole | 25 | 50 |
| Pre-gelatinized starch | 12.5 | 12.5 |
| Microcrystalline cellulose | 205 | 180 |
| Silicon dioxide | 0.625 | 0.625 |
| Sodium lauryl sulfate | 1.25 | 1.25 |
| Croscarmellose | 2.5 | 2.5 |
| Magnesium stearate | 2 | 2 |
| FDC Yellow #2 lake | 1.25 | 1.25 |

The active ingredient is passed through a suitable sieve and blended with the non-lactose excipients (except magnesium stearate) until a uniform blend is formed. The dry blend is screened and blended briefly with magnesium stearate. The resulting powder blend is then compressed into tablets of desired shape and size. Tablets of other strengths may be prepared by altering the ratio of the active ingredient (i.e., norastemizole) to the excipients or modifying the tablet weight.

### EXAMPLE 7

### Norastemizole Capsules (30 mg) with 120 mg of Sustained Release Pseudoephedrine HCL

| | Quantity |
|---|---|
| Component | (mg/capsule) |
| Norastemizole, Milled | 30.0 |
| Microcrystalline Cellulose NF, | 90.0 |
| Avicel PH101 | |
| Pregelatinized Starch, NF | 72.6 |
| Croscarmellose Sodium, NF | 7.0 |
| Magnesium Stearate, NF | 0.4 |
| Total Norastemizole Powder Blend | 200.0 |
| Pseudoephedrine HCl Diffucaps, | 204.0** |
| USP* | |
| Total | 404.0 |

*Diffucaps are sustained release beads of pseudoephedrine HCl manufactured by Eurand International
**204.0 mg of Diffucaps is equivalent to ca. 120 mg of pseudoephedrine HCl. Accordingly, the fill weight of Diffucaps beads is adjusted in each batch to account for the measured potency of pseudoephedrine HCl.

The active ingredients are sieved and blended with the excipients listed. The mixture is filled into suitably sized two-piece hard gelatin capsules using suitable machinery and methods well known in the art. See Remington's Pharmaceutical Sciences, 16th or 18th Editions, each incorporated herein in its entirety by reference thereto.

### EXAMPLE 8

### Rapid Dissolving Tablet, 15 mg and 30 mg

| | 30 | 15 |
|---|---|---|
| Component | mg/tablet | mg/tablet |
| Coated Pellets | | |
| Norastemizole | 32.18 | 16.09 |
| Neutres 250/350 | 56.80 | 28.25 |
| Pharmacoat 603¹ | 7.95 | 3.98 |
| Eudragit E100² | 9.69 | 4.85 |
| Syloid 244³ | 0.48 | 0.24 |
| 95% Ethanol* | | |
| Unit weight of coated | 107.1 | 53.4 |
| Norastemizole | | |
| Tablet | | |
| Coated Norastemizole pellets | 107.1** | 53.4** |
| Mannitol 60 | 206.9 | 103.5 |
| Mannitol 300 | 206.2 | 103.1 |
| Crospovidone | 61.2 | 30.6 |
| Aspartame | 12.2 | 6.1 |
| Syloid 244 | 3.1 | 1.6 |
| Magnesium Stearate | 12.2 | 6.1 |
| Flavor | 3.1 | 1.5 |
| Unit weight | 612.0 | 305.9 |

*Ethanol is a solvent used in layering and film coating and essentially all is removed in drying the granules.
**This is the quantity of pellets calculated to contain 30 mg of norastemizole according to pellet potency values. This quantity will vary from batch to batch.
¹Commercially available from Shim-Etsu Chemical Co. Ltd. of Tokyo, Japan.
²Commercially available from of Rohm and Hass of Philadelphia, PA.
³Commercially available from of W.R. Grace and Co. of Columbia, MD.

The coated norastemizole particles are passed through a suitable sieve and blended with the excipients (except magnesium stearate) until a uniform blend is formed. The dry blend is screened and blended briefly with magnesium stearate. The resulting powder blend is then compressed into tablets of desired shape and size.

### EXAMPLE 9

### Norastemizole Syrup (3 mg/mL)

Amount (mg) per mL

| | Bubble Gum | Grape Flavored |
|---|---|---|
| Component | Flavored Syrup | Syrup |
| Norastemizole | 3.0 | 3.0 |
| Acesulfame K, FCC | 5.0 | 5.0 |
| Sucralose, NF | 10.0 | 10.0 |
| Citric Acid | 10.0 | 10.0 |
| Monohydrate, USP | | |
| Sodium Citrate | 7.0 | 7.0 |
| Dihydrate, USP | | |
| Benzoic Acid, USP | 1.0 | 1.0 |
| Xylitol, NF | 300.0 | 300.0 |
| Sorbitol Solution, NF | 100.0 | 100.0 |
| Glycerin, USP | 100.0 | 100.0 |
| Bubble Gum Flavor | 2.5 | NA |
| Fruity Flavor | 1.5 | NA |
| Cream Flavor | 2.0 | NA |
| Grape Flavor | NA | 2.5 |
| “Bitter-Mask” Flavor | 2.5 | 2.5 |
| Purified Water, USP | qs to 1 mL | qs to 1 mL |

NA = Not applicable

While the present invention has been described with respect to the particular embodiments, it will be apparent to those skilled in the art that various changes and modifications may be made without departing from the spirit and scope of the invention as defined in the claims.

What is claimed is:

1. A solid pharmaceutical composition comprising norastemizole, or a pharmaceutically acceptable salt thereof; a diluent; a binder; a disintegrant; and a lubricant; wherein the diluent, binder, disintegrant, and lubricant are not the same.

2. The solid pharmaceutical composition of claim 1, wherein the composition is free of lactose.

3. The pharmaceutical composition of claim 1, wherein the disintegrant is present in an amount of from about 0.5 to 15 percent by weight of the pharmaceutical composition, and the lubricant is present in an amount of up to about 1 percent by weight of the pharmaceutical composition.

4. The solid pharmaceutical composition of claim 1 in a tablet or capsule dosage form.

5. The solid pharmaceutical composition of claim 1, wherein the norastemizole, or a pharmaceutically acceptable salt thereof, is present in an amount of from about 1 mg to 200 mg.

6. The solid pharmaceutical composition of claim 1, wherein the norastemizole, or a pharmaceutically acceptable salt thereof, is present in an amount of from about 2 mg to 100 mg.

7. The solid pharmaceutical composition of claim 1, further comprising pseudoephedrine, or a pharmaceutically acceptable salt thereof.

8. The solid pharmaceutical composition of claim 7, wherein the pseudoephedrine, or a pharmaceutically acceptable salt thereof, is adapted for sustained release.

9. A solid pharmaceutical composition comprising norastemizole, or a pharmaceutically acceptable salt thereof; microcrystalline cellulose; pregelatanized starch; croscarmellose sodium; and magnesium stearate.

10. The solid pharmaceutical composition of claim 9, wherein the norastemizole, or a pharmaceutically acceptable salt thereof, is present in an amount of from about 1 to 50 percent; the microcrystalline cellulose is present in an amount of from about 20 to 90 percent; the pregelatanized starch is present in an amount of from about 5 to 75 percent; the croscarmellose sodium is present in an amount of from about 1 to 5 percent; and the magnesium stearate is present in an amount of from about 0.05 to 0.8 percent by weight of the pharmaceutical composition.

11. The solid pharmaceutical composition of claim 9 in a tablet or capsule dosage form.

12. The solid pharmaceutical composition of claim 9, wherein the norastemizole, or a pharmaceutically acceptable salt thereof, is present in an amount of from about 1 mg to 200 mg.

13. The solid pharmaceutical composition of claim 12, wherein the norastemizole, or a pharmaceutically acceptable salt thereof, is present in an amount of from about 2 mg to 100 mg.

14. The solid pharmaceutical composition of claim 9, further comprising pseudoephedrine, or a pharmaceutically acceptable salt thereof.

15. The solid pharmaceutical composition of claim 14, wherein the pseudoephedrine, or a pharmaceutically acceptable salt thereof, is adapted for sustained release.

16. A solid pharmaceutical composition comprising (i) a therapeutically effective amount of coated particles of norastemizole, or a pharmaceutically acceptable salt thereof, wherein said particles are coated with an inert coating and (ii) a pharmaceutically acceptable excipient.

17. The solid pharmaceutical composition of claim 16, wherein the coated particles of norastemizole, or a pharmaceutically acceptable salt thereof, comprise granulated norastemizole particles, or a pharmaceutically acceptable salt thereof, and a pharmaceutically acceptable excipient.

18. The solid pharmaceutical composition of claim 16, wherein the inert coating comprises an inert film-forming agent in a solvent.

19. The solid pharmaceutical composition of claim 18, wherein the inert film-forming agent is methylcellulose, hydroxymethyl cellulose, carboxymethyl cellulose, hydroxypropylmethylcellulose, hydroxypropyl cellulose, hydroxyethylcellulose, methylhydroxyethylcelluose, sodium carboxymethylcellulose, or a mixture thereof.

20. The solid pharmaceutical composition of claim 18, wherein the inert film-forming agent is cross-linked ethylcellulose.

21. The solid pharmaceutical composition of claim 16 adapted as a quick dissolving dosage form.

22. A solid pharmaceutical composition comprising norastemizole, or a pharmaceutically acceptable salt thereof; a diluent; a binder; a disintegrant; and a lubricant; wherein the disintegrant is a super disintegrant.

23. The solid pharmaceutical composition of claim 22, wherein the super disintegrant is croscarmellose sodium or sodium starch glycolate.

24. The solid pharmaceutical composition of claim 23, wherein the super disintegrant is croscarmellose sodium.

25. A method of treating an allergic disorder in a mammal comprising administering to a mammal in need of treatment a therapeutically effective amount of the composition of claim 1.

26. The method of claim 25, wherein said mammal is a human.

27. The method of claim 25, wherein said allergic disorder is allergic rhinitis.

28. The method of claim 25, wherein said allergic disorder is solar uticaria or symptomatic dermographism.

29. A method of treating an allergic disorder in a mammal comprising administering to a mammal in need of treatment a therapeutically effective amount of the composition of claim 9.

30. The method of claim 29, wherein said mammal is a human.

31. The method of claim 29, wherein said allergic disorder is allergic rhinitis.

32. The method of claim 29, wherein said allergic disorder is solar uticaria or symptomatic dermographism

33. A method of treating an allergic disorder in a mammal comprising administering to a mammal in need of treatment a therapeutically effective amount of the composition of claim 16.

34. The method of claim 33, wherein said mammal is a human.

35. The method of claim 33, wherein said allergic disorder is allergic rhinitis.

36. The method of claim 33, wherein said allergic disorder is solar uticaria or symptomatic dermographism

37. A method of treating an allergic disorder in a mammal comprising administering to a mammal in need of treatment a therapeutically effective amount of the composition of claim 22.

38. The method of claim 37, wherein said mammal is a human.

39. The method of claim 37, wherein said allergic disorder is allergic rhinitis.

40. The method of claim 37, wherein said allergic disorder is solar uticaria, symptomatic dermographism.
